(19) United States
(12) Patent Application Publication
(10) Pub. No.: US 2011/0301392 A1
(43) Pub. Date: Dec. 8, 2011

(54) VARIATION OF TIN IMPREGNATION OF A CATALYST FOR ALKANE DEHYDROGENATION

(75) Inventors: Muhammad Iqbal Mian, Dortmund (DE); Max Heinritz-Adrian, Muenster (DE); Sascha Wenzel, Bochum (DE); Oliver Noll, Castrop-Rauxel (DE); Meinhard Schwefer, Meschede (DE); Helmut Gehrke, Bergkamen (DE)

(73) Assignee: UHDE GMBH, Dortmund (DE)

(21) Appl. No.: 13/140,768

(22) PCT Filed: Dec. 15, 2009
(86) PCT No.: PCT/EP2009/008976

§ 371 Date: Aug. 2, 2011

(30) Foreign Application Priority Data
Dec. 18, 2008 (DE) 10 2008 062 782.8
Dec. 3, 2009 (DE) 10 2009 056 539.6

(51) Int. Cl. C07C 5/48; B01J 21/02; B01J 21/04; B01J 21/06; B01J 37/08; B01J 23/14; B01J 21/10; B01J 23/02; B01J 37/02; B01J 35/02; B01J 21/08

(52) U.S. Cl. 585/319; 502/100; 502/342; 502/355; 502/340; 502/349; 502/232; 502/352; 502/242; 585/658; 585/440; 585/621; 585/324; 585/326

(57) ABSTRACT

A catalyst for the dehydrogenation of alkanes or alkyl substituents of hydrocarbons, is a shaped body having at least one oxide from the elements of the main or secondary group II to IV of the periodic table or of a mixed oxide thereof serving as base material of the shaped body. The catalyst further contains an additional constituent which is an oxide of an element of the main group IV of the periodic table, added during the shaping process. A platinum compound and a compound of an element of the main group IV of the periodic table is used as a surface constituent of the catalyst. The invention further relates to the production of the catalyst and to a method for the dehydrogenation of alkanes using the catalyst.

The invention relates to a catalyst, the production of a catalyst and a process using this catalyst for the dehydrogenation of alkanes or alkyl substituents of hydrocarbons.

The dehydrogenation of hydrocarbons is normally carried out in reactors the interior of which is equipped with a supporting device with a suitable catalyst and where a reaction gas mixture of hydrocarbons circulates around the catalyst. To ensure a conversion that is as efficient as possible, the catalyst is to be designed in such a way that it provides as large a surface as possible for the circulating gas mixture.

A catalyst is a solid designed, for example, in the form of cylinders, spheres or foams or of any other suitable form. The moulded body may also contain catalytic substances for the dehydrogenation of hydrocarbons. To achieve a high catalyst activity, additional catalytic substances will be applied onto the surface of the moulded bodies by various processes.

A catalyst is generally produced by various processes. First, the moulded body is produced, the selected solids, after having been ground/mixed, passing a moulding process such as sintering, pelletising, tabletting, prilling or extrusion. Depending on the moulding process, other process steps, such as drying and calcining, may be applied. Solutions containing catalytic materials may be applied onto the moulded body by, for example, impregnation; this process step may be repeated if requested. Normally, the impregnation step is followed by other steps as for example, drying, calcining, washing and re-drying.

Patent specification EP 0559 509 B1 describes a process for the dehydrogenation of aliphatic saturated hydrocarbons, a dehydrogenation catalyst being used which at least comprises an oxide of an element of the groups IIA, IIB, IIIA, IIIB, IVA and IVB of the periodic table, at least a noble metal of the platinum group, at least an additional metal from an element of groups VIIB or IVA and at least an alkali metal or alkaline earth metal. The catalyst also contains halogenated compounds and sulphur. In the dehydrogenation process, the flow leaving the dehydrogenation reaction is dried and fed to a separator, a liquid phase of non-converted hydrocarbons being mixed with the products to obtain a gaseous phase rich in hydrogen.

U.S. Pat. No. 5,151,401 A describes the production of a catalyst consisting of zinc aluminate and impregnated with a catalytically active substance made of a platinum compound. Suitable platinum compounds are, for example, platinum(II) chloride, platinum(IV) chloride, hexachloroplatinic acid or ammonium hexachloroplatinate. Preference is given to hexachloroplatinic acid. As the catalyst will contain chloride ions after the impregnation and calcining steps, the impregnation step is followed by a washing process. Chloride ions on the catalyst are not wanted since their corrosive nature may lead to the damage of plant sections during the reaction. Deionised water is used as washing solution. For stability improvement the carrier may be stabilised with calcium oxide, graphite, stearic acid or polyethylene.

Catalysts have the property of lowering the activation energy of the educts involved in a chemical reaction and thus accelerating the chemical reaction. In practice, however, the catalysts used become ineffective by secondary reactions after a specific period of time, inevitably resulting in a reduction of the reaction yield. In the catalytic dehydrogenation of alkanes, methane, ethane, carbon dioxide and other undesired by-products form after a certain reaction time, which later have to be separated from the product flow in time-consuming processes. Another by-product is the coke forming on the catalyst, thus considerably affecting the catalyst activity. Therefore, many state-of-the-art measures have been taken to increase the selectivity of the catalyst, thus suppressing the formation of by-products as completely as possible and extending the service life of the catalyst.

For example, the article “Use of Al₂O₃—SnO₂ as a support of Pt for selective dehydrogenation of light paraffins” in Catalysis Today 133-135 (2008) 28-34, by De Miguel describes a catalyst with aluminium oxide-tin dioxide (Al₂O₃—SnO₂) as the basic carrier to which tin is added as a surface component by means of precipitation from an aqueous tin chloride (SnCl₂) solution. This surface component is converted to metal oxide by oxidation. In the subsequent impregnation step, tin is also applied as surface metal simultaneously with platinum, the weight of the metal tin not exceeding 5% of the total weight.

The addition of an oxide compound of an element of the fourth main group of the periodic table results in a longer catalyst service life. This effect has been mentioned in the state of the art. GB 1346856 A describes a process for the dehydrogenation of alkanes in the presence of water vapour. The alkane to be dehydrogenated is directed over a catalyst applied onto a carrier made of zinc aluminate and tin dioxide and wetted with a compound of a metal from the VIII B group of the periodic table. Examples are the metals nickel, platinum, ruthenium, rhodium, palladium, osmium, iridium or mixtures thereof. For activation the catalyst may also contain compounds of the group of alkali metals, alkaline earth metals or germanium or tin compounds. Proof of the tin compounds contained in the catalyst is not described.

The aim of the invention therefore is to produce an efficient catalyst of higher selectivity and longer service life and to provide a process using this catalyst for the dehydrogenation of alkanes with reduced formation of by-products and higher selectivity of products as compared to the present state of the art.

The objective is achieved by using a catalyst for the dehydrogenation of alkanes or alkyl substituents of hydrocarbons, containing
a) a moulded body consisting of at least one or several oxides from an element of the second to fourth main group or subgroup of the periodic table or of a mixed oxide compound made up of the latter, the constituents serving as the basic material of the moulded body;
b) an additional component containing an oxide from an element of the fourth main group of the periodic table, the said oxide being added during the moulding process;
c) an active surface component containing a platinum compound;
d) an additional surface component containing a compound of an element of the fourth main group of the periodic table.

The invention especially claims a catalyst for the performance of catalytic alkane dehydrogenation, the catalyst being based on a moulded body. The moulded body consists of at least one or several oxides from an element of the second to fourth main group or subgroup of the periodic table or of a mixed oxide compound made up of the latter. This mixture of compounds serves as basic materials of the moulded body. The content of the basic materials may be more than 90% of the catalyst constituents. The additional component selected from an oxide of an element of the fourth main group of the periodic table with a small content in the catalyst of 0.1% to 4% is added during the moulding process. The catalyst according to the invention is completed by the additional catalytically active substances from a platinum compound and by a compound of an element of the fourth main group of the periodic table as a surface component.

Zinc oxide with aluminium oxide (zinc aluminate) is a preferred basic material for the moulded body of the catalyst for the dehydrogenation of alkanes or alkyl substituents of hydrocarbons. This compound, for example, may be produced by a calcination process of zinc oxide and aluminium oxide in a high-temperature furnace and constitutes the major constituent amount of the catalyst with more than 50%. The zinc aluminate compound, for example, may also be produced by a precipitation reaction from an aqueous or alcoholic mixture of a zinc salt solution with an aluminium salt solution. Moulded bodies made up of aluminium oxide, calcium oxide, zinc oxide, zirconium dioxide, magnesium dioxide or silicon dioxide as a main constituent are also suitable. The moulded body material may also consist of mixed phases of selected substances of the above-mentioned list. Of course, a combination of the substances may be used as moulded body material within the framework claimed above.

As additional component, i.e. an oxide from an element of the fourth main group of the periodic table, tin dioxide is given preference. Although the additional component features a low concentration in the moulded body, the said component can be recognised by the characteristic reflection angles of 26.6°, 33.8° and 51.7° when performing an X-ray diffraction with the wave length of CuK_{α}. By this additional compound combined with the basic compound tin dioxide is uniformly distributed over the entire moulded body.

Catalytically active surface components on the moulded body additionally increase the service life of the catalyst in operation, preference being given, on the one hand, to the platinum compound with a mass percentage of 0.01 to 1.0 of platinum and, on the other hand, to tin in the form of a compound of an element of the fourth main group of the periodic table with a mass percentage of 0.1 to 4.0. However, the additional surface component may also be germanium.

The invention claims a process for the production of the catalyst for the dehydrogenation of alkanes or alkyl substituents of hydrocarbons, the moulded body of the catalyst being impregnated in one or more impregnation steps simultaneously or consecutively with the claimed active and the additional surface component and, in subsequent process steps, the moulded body produced being further processed to obtain the catalyst.

First, the solid raw materials of the main constituent of the moulded body of at least one or several oxides from an element of the second to fourth main group or subgroup of the periodic table or of a mixed oxide compound made up of the latter, and a small content of the additional component, namely an oxide from an element of the fourth main group of the periodic table, are ground, mixed with binders and subjected to the moulding process to obtain the moulded body. Suitable moulding processes are, for example, sintering, pelletising, tabletting, prilling or extrusion processes, the optimum form of the moulded body being selected depending on the catalyst supporting device and/or the reactor.

After the moulding process, the moulded body must be calcined or dried if required. Not till then the active and additional catalytically active surface components can be simultaneously or consecutively applied onto the moulded body by means of impregnation, precipitation or immersion, for example, in the form of salt in an aqueous solution. The process steps may be repeated if required.

In an advantageous embodiment of the process for the production of the catalyst preference is given to an oxide compound for the moulded body, tin dioxide and one or several substances from the group of substances aluminium oxide, calcium oxide, zirconium dioxide, zinc oxide, silicon dioxide, magnesium oxide or other appropriate substances being suitable. The solids of the oxide compounds are powdered, mixed with binders and subjected to a moulding process. Other favoured variants for the moulded body are a water-soluble tin salt and one or several water-soluble salts of the metals aluminium, zinc, calcium or magnesium. The aqueous or alcoholic solutions are, if required, mixed with deionised water, neutralised and precipitated. After precipitation, the material obtained is filtered, dried and processed to the desired moulded body by a suitable moulding process. Typically, well suited moulding processes are tabletting or extrusion. The decision on the moulding process type is left to the person skilled in the art. Normally, it is the objective to produce an abrasion-proof moulded body with a sufficiently high porosity.

Optimally for the catalyst, its moulded body is treated with catalytically active substances. A platinum compound especially suitable for impregnation is hexachloroplatinic acid or its salts. Of course, other soluble platinum compounds such as platinum(II) halogenides and platinum(IV) halogenides may be used as well. A water-soluble tin compound such as tin chloride or tin nitrate is usually used for the impregnation with the additional surface component, a compound of an element of the fourth main group of the periodic table. Both an aqueous solution and an ethanol or methanol solution containing the surface component may be used for impregnation. The impregnation of the moulded body with the specified surface components in solutions may be carried out consecutively or simultaneously.

Typically, the impregnation is carried out by spraying or immersion the moulded body with the solution containing the catalytically active substances. In principle, other processes ensuring a uniform distribution of the substances for impregnation of the moulded body are also suitable as impregnation processes.

After impregnation the moulded body passes the subsequent process steps of calcination, washing and/or drying as required. Some process steps may also be repeated. Then, the desired catalyst is finished.

A process for the dehydrogenation of alkanes or alkyl substituents of hydrocarbons is also claimed, an alkane or hydrocarbon to be dehydrogenated being routed in a mixture with quasi-inert gases through a reactor for dehydrogenation charged with the catalyst according to the invention. For this, the usual general parameters of the alkane dehydrogenation are to be applied.

In a preferred embodiment the dehydrogenation of alkanes is carried out at a temperature of 480° C. to 820° C. The reaction gives the desired alkene and hydrogen, the alkene being discharged and non-converted alkane and water vapour being redirected through the reactor. This reaction step is preferably performed in an adiabatic process or allothermic process with external heating. However, in principle, any process and/or device able of performing such a dehydrogenation reaction is suitable. For example, water vapour, carbon dioxide or nitrogen are suitable as quasi-inert gases. In some processes, it is also customary to add hydrogen for suppressing the formation of coke.

If the process is carried out with the catalyst produced according to the invention, higher conversion rates and thus an increased reaction rate will be achieved depending on the reaction performed. However, in particular, a higher selectivity is obtained, corresponding to a reduced formation of by-products. As a result, fewer catalysts are required. The catalyst according to the invention also has a considerably longer service life. This also contributes to lower operating costs of the entire process.

It is also possible to combine the process step of alkane dehydrogenation with a subsequent process step of hydrogen combustion and to use the catalyst according to the invention. In doing so, the hydrogen, on the one hand, is withdrawn from the equilibrium and shifted to the desired direction, on the other hand, heat is generated, causing the gas to be redirected through the dehydrogenation reactor without any further heating, thus allowing non-converted alkane to react. This process step as well is preferably carried out at a temperature of 480° C. to 820° C.

In a further embodiment of the process with the catalyst according to the invention, the hydrogen is oxidised at a temperature of 480° C. to 820° C. As hydrogen combustion takes place exothermically, the heat being generated in this process step may be used for the subsequent endothermic dehydrogenation.

Propane, n-butane or i-butane are frequently used as basic materials for producing propene or n-butene or isobutene. Ethyl benzene or single olefins such as n-butene may also be used as compounds to be dehydrogenated. In this case, styrene or 1,3-butadiene are obtained. Finally higher alkanes may, for example, be dehydrogenated by the process according to the invention. All hydrocarbons mentioned may well be dehydrogenated using the catalyst and the process according to the invention.

Nevertheless, on account of slight but continuous coking the catalyst must be regenerated at regular intervals. This is normally done by transferring an oxygen-containing gas, the carbon-containing deposits on the catalyst being combusted.

In the following the invention is explained in a drawing on the basis of an embodiment example, in this case a propane dehydrogenation according to the process illustrated in WO 2006050957 A1. All catalysts used have been impregnated with solutions having a platinum content of 0.6%. The conversion rate of propane and the selectivity of propene are illustrated in the figure. Four different catalysts are compared to each other, two catalysts each being based on the same moulded body which differs in its tin content only.

The first moulded body serving as the basis for variants (1) and (2) has a tin content of 0.95%. Variant (1) does not contain any tin by additional impregnation, variant (2) has a tin content of 0.48% applied together with platinum. Variant (2) compared to variant (1) shows a higher selectivity. The second moulded body serving as the basis for variants (3) and (4) does not contain any tin. Variant (3) with an impregnated-tin content of 0.95% corresponding to the tin content of variant (1) also shows, when compared to the latter, a considerable increase in selectivity at the same conversion rate, but it remains below the selectivity of variant (2). An increase in the tin content applied by impregnation only as shown in variant (4) results in a lower selectivity as compared to variants (2) and (3). Therefore, maximum selectivities can only be achieved if the moulded body contains tin dioxide and is additionally impregnated with platinum and tin.

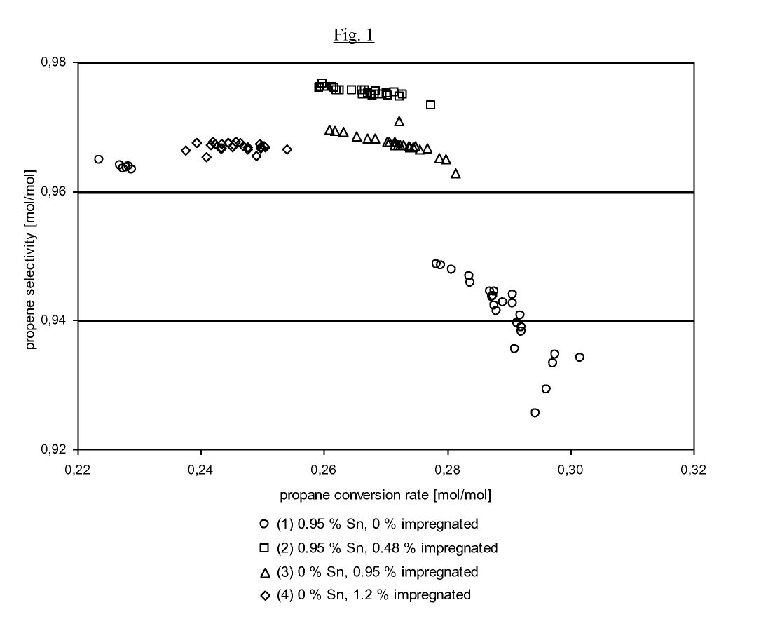

It can be derived from the graph in FIG. 1 that the catalyst which contains 0.95% tin in the moulded body and also 0.48% tin applied by impregnation with a tin-containing solution results in a considerably higher selectivity of propene as compared to catalysts which only contain tin in the moulded body or only contain tin applied by impregnation. The above example gives an excellent illustration of the interaction of the catalyst according to the invention, consisting in a combination of the tin-containing moulded body and the impregnation with a tin solution, in the dehydrogenation of hydrocarbons.

1. A catalyst for the dehydrogenation of alkanes or alkyl substituents of hydrocarbons, comprising:
a) a moulded body consisting of at least one oxide from an element of the second to fourth main group or subgroup of the periodic table or a mixed oxide thereof serving as the base material of the moulded body;
b) an additional component containing an oxide from an element of the fourth main group of the periodic table, the additional component being added during the moulding process used to form the moulded body;
c) an active surface component containing a platinum compound;
d) an additional surface component containing a compound of an element of the fourth main group of the periodic table.

2. The catalyst of claim 1, wherein more than 50% of the base material of the moulded body consist of zinc aluminate as a mixed oxide compound.

3. The catalyst of claim 1, wherein more than 50% of the base material of the moulded body consist of aluminium oxide, magnesium oxide, calcium oxide, zirconium dioxide or silicon dioxide or a combination thereof.

4. The catalyst of claim 1, wherein the additional component is tin dioxide having the characteristic reflection angles of 26.6°, 33.8° and 51.7° when performing an X-ray diffraction with the wave length of CuKₐ.

5. The catalyst of claim 1, wherein the additional surface component contains tin.

6. The catalyst of claim 1, having a tin content of 0.1 to 4.0 weight percent based on the total weight of the catalyst.

7. The catalyst of claim 1, wherein the content of platinum 0.01 to 1.0 weight percent based on the total weight of the catalyst.

8. A process for the production of a catalyst of claim 1, comprising:
a) molding at least one oxide from an element of the second to fourth main group or subgroup of the periodic table or a mixed oxide thereof as a basic material to form a moulded body;
b) adding an additional component containing an oxide from an element of the fourth main group of the periodic table during the moulding process,
c) simultaneously or consecutively impregnating the moulded body in one or more impregnation steps with the active surface component and the additional surface component, and
(d) further processing the moulded body in subsequent process steps to obtain the catalyst.

9. The process of claim 8, wherein the process steps for producing the catalyst include impregnation, drying, precipitating, washing and calcining.

10. The process of claim 8, wherein tin dioxide and one or more substances selected from the group consisting of aluminium oxide, calcium oxide, zirconium dioxide, silicon dioxide and magnesium oxide are used, and the moulded body is produced by grinding of the solid raw materials, mixing and calcining.

11. The process of claim 8, wherein a water-soluble tin salt and one or more water-soluble salts of the metals aluminium, zinc, calcium or magnesium are used and the moulded body is produced by mixing with deionised water, neutralizing, precipitating, drying and calcining.

12. The process of claim 8, wherein the moulding process is a sintering process, a pelletising process, a tabletting process, an extrusion process or a reticulation process.

13. A process for the dehydrogenation of alkanes, comprising routing an alkane or hydrocarbon to be dehydrogenated in a mixture with quasi-inert gases for dehydrogenation through a reactor charged with the catalyst of claim 1 and dehydrogenating the alkane or hydrocarbon.

14. The process of claim 13, wherein the alkane is routed for dehydrogenation in a mixture with water vapour through a reactor charged with the catalyst in a first step, to form a gas mixture comprising residual alkane, alkene, hydrogen and water vapour, and feeding the gas mixture formed in the first step to another reactor in a second step, the reactor in the second step also charged with the catalyst, the hydrogen contained in the mixture being oxidised in the second step.
